(19) United States
(12) Patent Application Publication
(10) Pub. No.: US 2019/0203289 A1
(43) Pub. Date: Jul. 4, 2019

(54) GENE SEQUENCING CHIP AND SEQUENCING METHOD THEREOF, AND GENE SEQUENCING DEVICE

(71) Applicants: BEIJING BOE OPTOELECTRONICS TECHNOLOGY CO., LTD., Beijing (CN); BOE TECHNOLOGY GROUP CO., LTD., Beijing (CN)

(72) Inventors: Fengchun PANG, Beijing (CN); Chuncheng CHE, Beijing (CN); Hailin XUE, Beijing (CN); Xibin SHAO, Beijing (CN); Peizhi CAI, Beijing (CN)

(21) Appl. No.: 16/325,235

(22) PCT Filed: Mar. 30, 2018
(86) PCT No.: PCT/CN2018/081344

§ 371 Date: Feb. 13, 2019

(30) Foreign Application Priority Data
Jul. 31, 2017 (CN) 201710639963.X

(51) Int. Cl. C12Q 1/6869; C12Q 1/6837; B01L 3/00; H01G 9/20; B01J 19/02

(52) U.S. Cl. CPC C12Q 1/6869; C12Q 1/6837; B82Y 40/00; H01G 9/2031; B01J 19/02; B01L 3/5027

(57) ABSTRACT

A gene sequencing chip, a sequencing method thereof and a gene sequencing device are provided. The gene sequencing chip includes: a first substrate having a first surface; at least one recessed portion recessed from the first surface into the first substrate; a graphene oxide layer located at a bottom of the recessed portion; and a first electrode and a second electrode which are electrically connected with the graphene oxide layer. The recessed portion is configured to receive a sample to be tested, and the first electrode and the second electrode are configured to detect a resistance of the graphene oxide layer.

The application claims priority to the Chinese patent application No. 201710639963.X, filed on Jul. 31, 2017, the entire disclosure of which is incorporated herein by reference as part of the present application.

### TECHNICAL FIELD

At least one embodiment of the present disclosure relates to a gene sequencing chip, a sequencing method thereof and a gene sequencing device.

### BACKGROUND

With continuous development of gene sequencing technology, gene sequencing technology has gradually become the most commonly used technology in modern molecular biology research with a wide range of application scenarios. Therefore, the device for gene sequencing has a large market space.

Since the development of the first generation of gene sequencing in 1977, gene sequencing technology has made great progress and includes the first generation of sanger sequencing technology, the second generation of high-throughput sequencing technology, the third generation of single-molecule sequencing technology, and the fourth generation of nano-holes sequencing technology. The current mainstream sequencing technology is still based on the second generation of high-throughput sequencing.

The second generation of high-throughput sequencing technology mainly includes Illumina's sequencing by synthesis technology, Thermo Fisher's ion semiconductor sequencing technology, ligation sequencing technology and Roche's pyro-sequencing technology, among which Illumina's sequencing by synthesis technology has advantages of ultra-high throughput and relative long read, thus has a market share of over 70%.

A conventional gene sequencing technology performs modifications on various bases by different fluorescence groups. When these bases are paired with the gene fragment to be tested, the fluorescent group is released; at this time, the type of the base can be determined by detecting the color of fluorescence by an optical system, thereby obtaining the sequence of the gene fragment to be tested.

### SUMMARY

At least one embodiment of the present disclosure provides a gene sequencing chip, including: a first substrate having a first surface; at least one recessed portion recessed from the first surface into the first substrate; a graphene oxide layer located at a bottom of the recessed portion; and a first electrode and a second electrode electrically connected with the graphene oxide layer, respectively; the recessed portion is configured to receive a sample to be tested, and the first electrode and the second electrode are configured to detect a resistance of the graphene oxide layer.

For example, in the gene sequencing chip provided by an embodiment of the present disclosure, the first electrode is not in direct contact with the second electrode.

For example, in the gene sequencing chip provided by an embodiment of the present disclosure, the first substrate includes: a sidewall of the recessed portion configured to define the recessed portion; and a first base substrate located on a side of the recessed portion away from the first surface and configured to support the sidewall of the recessed portion, the first electrode and the second electrode are located between the sidewall of the recessed portion and the first base substrate, the first electrode and the second electrode extend to a bottom of the recessed portion to be in contact with the graphene oxide layer, respectively.

For example, in the gene sequencing chip provided by an embodiment of the present disclosure, the first electrode includes a first contact portion extending to the recessed portion, the second electrode includes a second contact portion extending to the recessed portion, and the graphene oxide layer covers the first contact portion and the second contact portion.

For example, in the gene sequencing chip provided by an embodiment of the present disclosure, a shape of an orthographic projection of the recessed portion on the first substrate includes two opposite edges, and the first electrode and the second electrode are located between two opposite sidewalls of the recessed portion and the first base substrate, respectively.

For example, in the gene sequencing chip provided by an embodiment of the present disclosure, the first substrate further includes an insulation layer located on a side of the first base substrate close to the first surface, and the insulation layer is located on a side of the first electrode, the second electrode, and the graphene oxide layer away from the first surface.

For example, in the gene sequencing chip provided by an embodiment of the present disclosure, the recessed portion includes a microchannel or a micro-hole.

For example, in the gene sequencing chip provided by an embodiment of the present disclosure, the graphene oxide layer is provided in a one-to-one correspondence with the recessed portion.

For example, in the gene sequencing chip provided by an embodiment of the present disclosure, the at least one recessed portion includes a plurality of recessed portions, the plurality of recessed portions are arranged in an array, and graphene oxide layers in the recessed portions of a same column share one first electrode.

For example, in the gene sequencing chip provided by an embodiment of the present disclosure, the gene sequencing chip further includes a third electrode electrically connected with second electrodes in the recessed portions of a same row.

For example, in the gene sequencing chip provided by an embodiment of the present disclosure, the first substrate includes a plurality of connection via holes, and the third electrode is electrically connected with the second electrodes in the recessed portions of the same row through the plurality of connection via holes.

For example, in the gene sequencing chip provided by an embodiment of the present disclosure, the third electrode is located on the first surface.

For example, in the gene sequencing chip provided by an embodiment of the present disclosure, the gene sequencing chip further includes: a second substrate having a second surface, the second substrate and the first substrate are opposite to each other to form a cell, and the first surface is opposite to the second surface.

For example, in the gene sequencing chip provided by an embodiment of the present disclosure, the second substrate further includes: at least one flow channel, the at least one flow channel being located opposite to and in communication with the at least one recessed portion; a second base substrate located on a side of the flow channel away from the second base surface; and a flow channel sidewall located on a side of the second substrate close to the second surface, the flow channel sidewall being configured to define the flow channel.

At least one embodiment of the present disclosure further provides a gene sequencing device including any one of the gene sequencing chips as described above.

For example, in the gene sequencing device provided by an embodiment of the present disclosure, the gene sequencing device further includes: a first circuit board electrically connected with the first electrode; and a second circuit board electrically connected with the second electrode, the first circuit board is configured to apply a driving signal to the first electrode, the second circuit board is configured to detect a detection signal on the second electrode, or the second circuit board is configured to apply a driving a signal to the second electrode, the first circuit board is configured to detect a detection signal on the first electrode.

For example, in the gene sequencing device provided by an embodiment of the present disclosure, the gene sequencing chip includes any one of the gene sequencing chips as described above, the sequencing method includes: receiving a sample to be tested in the recessed portion; adding a fluorescently labeled deoxy-ribonucleoside triphosphate to the recessed portion; detecting a resistance of the graphene oxide layer through the first electrode and the second electrode.

### BRIEF DESCRIPTION OF THE DRAWINGS

In order to clearly illustrate the technical solution of the embodiments of the present disclosure, the drawings of the embodiments will be briefly described in the following; it is obvious that the described drawings are only related to some embodiments of the present disclosure and thus are not limitative of the present disclosure.

FIG. 1 is a schematic cross-sectional view of a gene sequencing chip according to an embodiment of the present disclosure;

FIG. 2 is a schematic cross-sectional view of another gene sequencing chip according to an embodiment of the present disclosure;

FIG. 3 is a schematic plan view of a gene sequencing chip according to an embodiment of the present disclosure;

FIG. 4 is a schematic plan view of another gene sequencing chip according to an embodiment of the present disclosure;

FIG. 5 is a schematic plan view of another gene sequencing chip according to an embodiment of the present disclosure;

FIG. 6 is a schematic cross-sectional view of a gene sequencing chip according to an embodiment of the present disclosure;

FIG. 7 is a schematic plan view of a gene sequencing device according to an embodiment of the present disclosure;

FIG. 8 is a flowchart of a sequencing method of a gene sequencing chip according to an embodiment of the present disclosure.

### DETAILED DESCRIPTION

In order to make objects, technical details and advantages of the embodiments of the present disclosure apparent, the technical solutions of the embodiments will be described in a clearly and fully understandable way in connection with the drawings related to the embodiments of the present disclosure. Apparently, the described embodiments are just a part but not all of the embodiments of the present disclosure. Based on the described embodiments herein, those skilled in the art can obtain other embodiment(s), without any inventive work, which should be within the protection scope of the present disclosure.

Unless otherwise defined, all the technical and scientific terms used herein have the same meanings as commonly understood by one of ordinary skill in the art to which the present disclosure belongs. The terms “first,” “second,” etc., which are used in the present disclosure, are not intended to indicate any sequence, amount or importance, but distinguish various components. Also, the terms “comprise,” “comprising,” “include,” “including,” etc., are intended to specify that the elements or the objects stated before these terms encompass the elements or the objects and equivalents thereof listed after these terms, but do not preclude the other elements or objects. The phrases “connect”, “connected”, etc., are not intended to define a physical connection or mechanical connection, but may include an electrical connection, directly or indirectly.

Conventionally, in sequencing, four kinds of deoxy-ribonucleoside triphosphates (dNTPs) with four kinds of fluorescent labels are added to the gene sequencing chip to react with the sample to be tested (e.g., DNA single strand or RNA) in the sequencing by synthesis technology. These dNTPs have a chemically cleaved azide group at the 3′ end, which allows only a single base to be bonded per cycle; then irradiating the gene sequencing chip with a laser to allow the fluorescence group of the deoxy-ribonucleoside triphosphate (dNTP) involved in reaction to emit fluorescence; finally, scanning the gene sequencing chip by a scanning device, such as a CCD image sensor, to record information of the above fluorescence, and converting the information of the fluorescence into a base sequence of the sample to be tested. However, the above-described process of sequencing by synthesis technology is complicated, and it is necessary to indirectly obtain a base sequence of a sample to be tested by analyzing an image, resulting in low efficiency. Moreover, it is necessary to purchase an additional camera device such as a CCD image sensor, resulting in high costs, which is not conducive to the promotion and utilization of gene sequencing technology.

Embodiments of the present disclosure provide a gene sequencing chip, a sequencing method thereof, and a gene sequencing device. The gene sequencing chip includes: a first substrate having a first surface; at least one recessed portion recessed from the first surface into the first substrate; a graphene oxide layer located at a bottom of the recessed portion; and a first electrode and a second electrode which are electrically connected with the graphene oxide layer. The recessed portion is configured to receive a sample to be tested, and the first electrode and the second electrode are configured to detect a resistance of the graphene oxide layer. In this case, whether the currently added fluorescently labeled deoxy-ribonucleoside triphosphate is reacted with a base in the sample to be tested can be determined by detecting a change in resistance of the graphene oxide layer through the first electrode and the second electrode in the gene sequencing chip, thereby directly converting the fluorescence information into an electrical signal for analysis, simplifying sequencing process, reducing sequencing time, and improving sequencing efficiency. Moreover, upon the gene sequencing chip being adopted for gene sequencing, an additional imaging device such as a CCD image sensor is not required, and the sequencing costs can be reduced.

Hereinafter, the gene sequencing chip, the sequencing method thereof and the gene sequencing device provided by the embodiments of the present disclosure are described with reference to the accompanying drawings.

An embodiment of the present disclosure provides a gene sequencing chip. FIG. 1 is a schematic cross-sectional view of a gene sequencing chip according to the embodiment. As illustrated in FIG. 1, the gene sequencing chip includes: a first substrate 110 having a first surface 101; at least one recessed portion 120 recessed from the first surface 101 into the first substrate 110; and a graphene oxide layer 130 located at a bottom of the recessed portion 120; and a first electrode 140 and a second electrode 150 which are electrically connected with the graphene oxide layer 130. The recessed portion 120 is configured to receive a sample to be tested, and the first electrode 140 and the second electrode 150 can detect a resistance of the graphene oxide layer 130. For example, the first electrode and the second electrode may be applied with a voltage difference therebetween and configured to detect a change in current on the graphene oxide layer, so that the resistance of the graphene oxide layer can be detected. It should be noted that the above-mentioned gene sequencing chip may not include the reagent involved in the reaction and the sample to be tested.

In the gene detection chip provided by the present embodiment, when a sample to be tested (for example, a single strand of DNA) is placed in the microstructure, and four fluorescently labeled deoxy-ribonucleoside triphosphates (dNTPs) are added to the microstructure, if a certain kind of fluorescently labeled deoxy-ribonucleoside triphosphate is base-paired with the sample to be tested, because both the fluorescent dye and the graphene oxide have a C═C—C═C conjugated double bond and the distance therebetween is very close, resulting in a stack of C═C—C═C, and fluorescence energy resonance and transfer, i.e., fluorescence quenching occurs. Upon fluorescence resonance and transfer occurring, photon energy generated by the fluorescent dye is absorbed by the graphene oxide, resulting in reducing the resistance of the graphene oxide. The gene sequencing chip can detect a change in resistance of the graphene oxide layer through the first electrode and the second electrode, so that whether the currently added fluorescently labeled deoxy-ribonucleoside triphosphate has a base paired reaction with the sample to be tested can be determined; thereby directly converting the fluorescence information into an electrical signal for analysis, which can simplify the sequencing process, reduce the sequencing time, and improve the sequencing efficiency. Moreover, upon the gene sequencing chip being adopted for gene sequencing, an additional imaging device such as a CCD image sensor is not required, and the sequencing costs can be reduced. It should be noted that the above-mentioned four different kinds of deoxy-ribonucleoside triphosphates include adenine deoxy-ribonucleoside triphosphate, uracil deoxy-ribonucleoside triphosphate, cytosine deoxy-ribonucleoside triphosphate, and guanine deoxy-ribonucleoside triphosphate.

It is worth noting that upon the gene sequencing chip provided by the embodiment being used for gene sequencing, a sample to be tested (for example, a DNA single strand) modified at both ends with amino groups is firstly added to the flow channel to allow the two ends of the sample to be tested to be fixedly connected with a surface of the graphene oxide, so that the sample to be tested can be amplified by polymerase chain reaction (PCR) on the surface of the graphene oxide layer. Of course, embodiments of the present disclosure include, but are not limited thereto. A gel film layer may be formed on the graphene oxide layer, and a linker is provided on the gel film layer, the sample to be tested is attached to the linker of the gel film layer in a paired manner, thereby fixing the sample to be tested in the recessed portion. The gel film layer may be made of a conventional material, for example, may be made of, a hydrogel; for further example, may be made of a substance having a colloidal structure, a substance of a polymer mesh structure, or a substance of a cross-linked polymer structure. For example, the substance having a colloidal structure includes agarose. For example, the substance of a polymer mesh structure includes gelatin. For example, the substance of the cross-linked polymer structure includes polyacrylamide. A material of the gel film layer may also be silane-free acrylamide or N-[5-(2-bromoacetyl)aminopentyl]acrylamide (BRAPA).

Dropping a graphene oxide solution onto the substrate and performing a dry process. Removing the graphene oxide between adjacent micro-holes by polishing, leaving graphene oxide only at the bottom of the micro-holes.

For example, a material of the first electrode and the second electrode may be selected from conductive materials such as indium tin oxide (ITO), gold, silver, copper, aluminum, and the like. The first electrode and the second electrode may be formed by firstly depositing a metal or metal oxide film and then patterning the metal or metal oxide film.

For example, in some examples, as illustrated in FIG. 1, the first electrode 140 is not in direct contact with the second electrode 150.

For example, in some examples, as illustrated in FIG. 1, the first substrate 110 includes: a sidewall 112 of the recessed portion configured to define the recessed portion 120, and a first base substrate 111. The first base substrate 111 is located on a side of the recessed portion 120 away from the first surface and is formed as the sidewall 112 of the recessed portion. The first electrode 140 and the second electrode 150 are located between the sidewall 112 of the recessed portion and the first base substrate 111 and extend to the bottom of the recessed portion 120 to be in contact with the graphene oxide layer 130, respectively; therefore, the first electrode and the second electrode are electrically connected with the graphene oxide layer, respectively. Moreover, because the first electrode and the second electrode are located between the sidewall of the recessed portion and the first base substrate, the first electrode and the second electrode can be prevented from being corroded by the sample or solution to be tested placed in the recessed portion. Of course, embodiments of the present disclosure include, but are not limited thereto, and the first electrode and the second electrode may be electrically connected with the graphene oxide layer by other means.

For example, a material of the first substrate may include silicon, glass, polyterpene terephthalate (PET), polymethyl methacrylate (PMMA), or the like.

For example, a material of the sidewall of the recessed portion may include silicon oxide, silicon nitride, or the like.

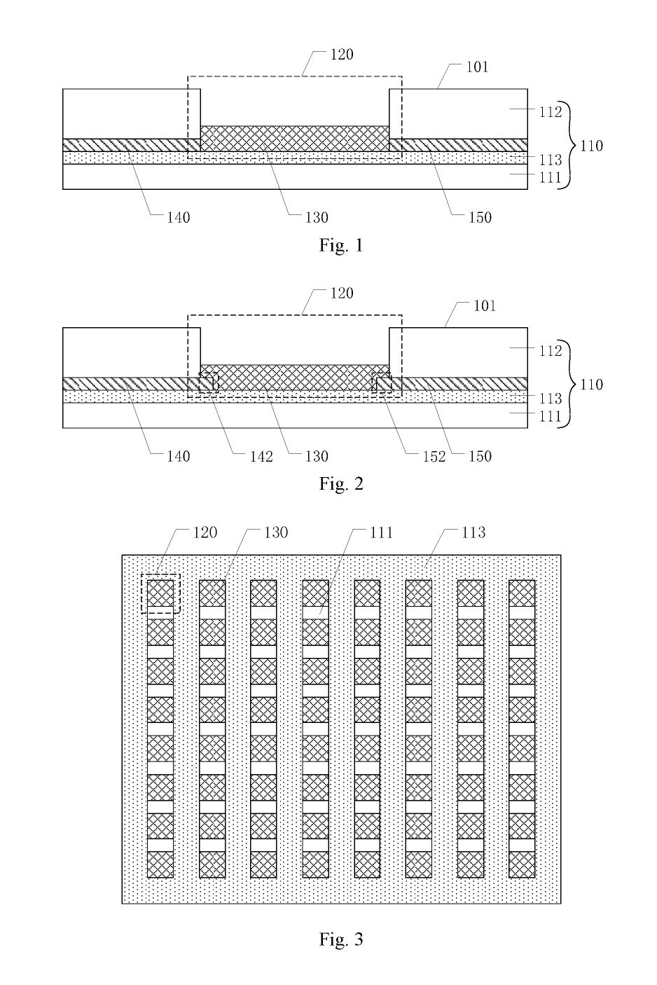

FIG. 2 is a schematic cross-sectional view of another gene sequencing chip according to an embodiment of the present disclosure. As illustrated in FIG. 2, the first electrode 140 includes a first contact portion 142 that extends to the recessed portion 120, the second electrode 150 includes a second contact portion 152 that extends to the recessed portion 120, and the first contact portion 142 and the second contact portion 152 are covered by the graphene oxide layer 130. That is, orthographic projections of the first contact portion 142 and the second contact portion 152 on the first base substrate 111 fall within an orthographic projection of the graphene oxide layer 130 on the first base substrate 111, so that the graphene oxide layer can form a strong electrical connection with the first electrode and the second electrode by covering the first contact portion and the second contact portion, and the graphene oxide layer can also play a role of protection to the first electrode and the second electrode to further prevent the first electrode and the second electrode from being corroded by the sample or solution to be tested in the recessed portion.

For example, in some examples, as illustrated in FIG. 2, a shape of an orthographic projection of the recessed portion 120 on the first substrate 110 includes two opposite edges, thus the recessed portion 120 has two sidewalls 112 of the recessed portion which are opposite to each other. The first electrode 140 and the second electrode 150 are respectively located between the two opposite sidewalls 112 of the recessed portion 120 and the first base substrate 111 so as to be electrically connected with two opposite ends of the graphene oxide layer 130 at the bottom of the recessed portion 120, respectively, so as to detect the change in resistance of the graphene oxide layer more accurately.

For example, in an example, as illustrated in FIGS. 1 and 2, the first substrate 110 further includes an insulation layer 113 provided between the first base substrate 111 and a structure of the first electrode 140, the second electrode 150, and the graphene oxide layer 130. That is, the insulation layer 113 is located on a side of the first base substrate 111 close to the first surface 101, and on a side of the structure of the first electrode 140, the second electrode 150, and the graphene oxide layer 130 away from the first surface 101. Of course, the embodiments of the present disclosure include, but are not limited thereto. The gene sequencing chip may be also formed without the insulation layer as described above, and the first electrode, the second electrode, and the graphene oxide layer may be directly provided on the first substrate.

FIG. 3 is a schematic plan view of a gene sequencing chip according to an embodiment. As illustrated in FIG. 3, the recessed portion 120 may be a microchannel. That is, the two sides of the recessed portion 120 are provided with sidewalls of the recessed portion, and the other two sides of the recessed portion 120 are not provided with sidewalls of the recessed portion. For example, as illustrated in FIG. 3, in the case that a plurality of recessed portions 120 are arranged in an array, the recessed portions 120 of the same column may constitute one channel, thereby facilitating circulation of various reaction reagents.

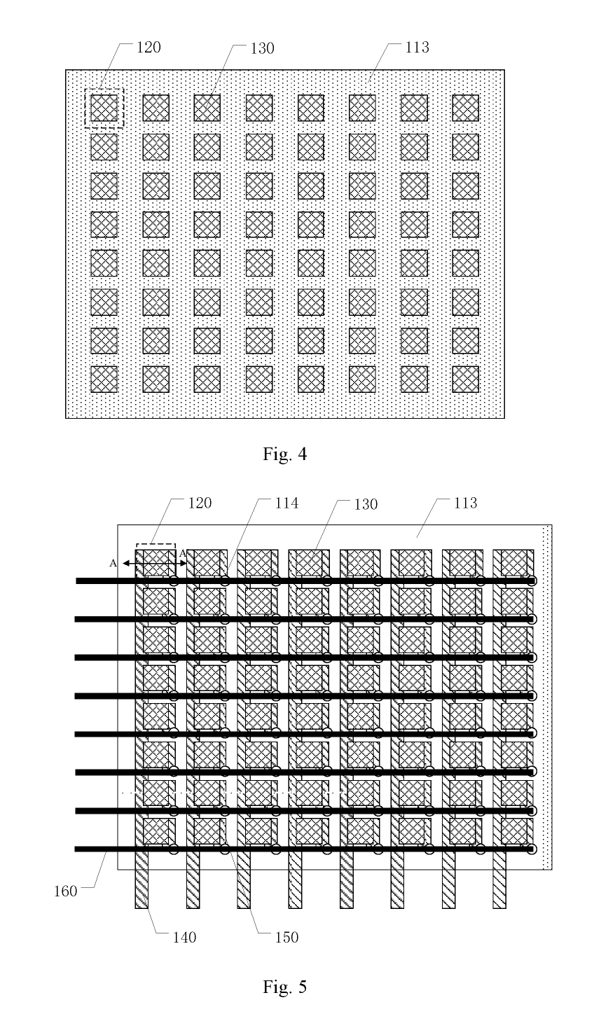

FIG. 4 is a schematic plan view of another gene sequencing chip according to an embodiment. As illustrated in FIG. 4, the recessed portion 120 may be a micro-hole. That is, a sidewall of the recessed portion is provided around the recessed portion 120, each recessed portion is independent, thereby reducing mutual interference between different recessed portions.

For example, as illustrated in FIG. 4, a planar shape of the recessed portion 120 may be a rectangle. Of course, embodiments of the present disclosure include, but are not limited thereto, and the planar shape of the recessed portion may further include a circular shape, an elliptical shape, or the like.

For example, in some examples, as illustrated in FIGS. 3 and 4, the graphene oxide layers 130 are arranged in a one-to-one correspondence with the recessed portions 120. That is to say, each of the recessed portions 120 is correspondingly provided with one graphene oxide layer 130, so that whether or not the base paired reaction occurs in the sample to be tested in the recessed portion can be determined by detecting a change in resistance of the graphene oxide layer in each recessed portion.

FIG. 5 is a plan view of another gene sequencing chip according to an embodiment (FIGS. 1 and 2 illustrate a cross-section taken along line AA′ in FIG. 5). As illustrated in FIG. 5, the at least one recessed portion 120 includes a plurality of recessed portions 120, and the plurality of recessed portions 120 are arranged in an array, and the graphene oxide layers 130 in the recessed portions 120 of the same column share one first electrode 140, so that an electrical signal can be applied to or received by the graphene oxide layers in the recessed portions of the same column through the first electrode at the same time, thereby simplifying electrode pattern of the gene sequencing chip, increasing density of the recessed portion, and improving sequencing throughput of the gene sequencing chip. It should be noted that when an electrical signal is simultaneously applied to or received by the graphene oxide layers in the recessed portions of the same column through the first electrode, the second electrode can be utilized to be applied with or receive an electrical signal on the above-mentioned graphene oxide in a time-dividing manner, so that the resistance of each of the above graphene oxide layers can be detected.

For example, in some examples, as illustrated in FIG. 5, the gene sequencing chip further includes a third electrode 160 electrically connected with the graphene oxide layers 130 in the recessed portions 120 of the same row, such as electrically connected by the second electrodes 140 of the same row, so that the graphene oxide layers 130 in the recessed portions 120 belonging to the same row can be connected by the third electrode 160, so that the graphene oxide layer in the recessed portions of the same row can be simultaneously applied with or receive an electrical signal by the second electrode, thereby simplifying electrode pattern on the gene sequencing chip, increasing density of the recessed portion, and improving sequencing throughput of the gene sequencing chip. It should be noted that upon the graphene oxide layers in the recessed portions of the same column being applied with or receiving an electrical signal simultaneously through the second electrode, the first electrode can be utilized to be applied with or receive an electrical signal on the above-mentioned graphene oxide in a time-dividing manner, so that the resistance of each of the above graphene oxide layers can be detected.

For example, in some examples, as illustrated in FIG. 5, the third electrode 160 is located on the first surface 101, and the first substrate 110 may be provided with a plurality of connection via holes 114, and the third electrode 160 may be electrically connected with the plurality of second electrodes 140 in the recessed portion 120 of the same row through the plurality of connection via holes. 114. Of course, embodiments of the present disclosure include, but are not limited thereto, and other methods for electrically connecting the third electrode with the graphene oxide layers in the recessed portion of the same row may be adopted.

It should be noted that the third electrode 160 may also be located between the first base substrate 111 and the insulation layer 113 and connected with the second electrodes 140 through via holes in the insulation layer 113.

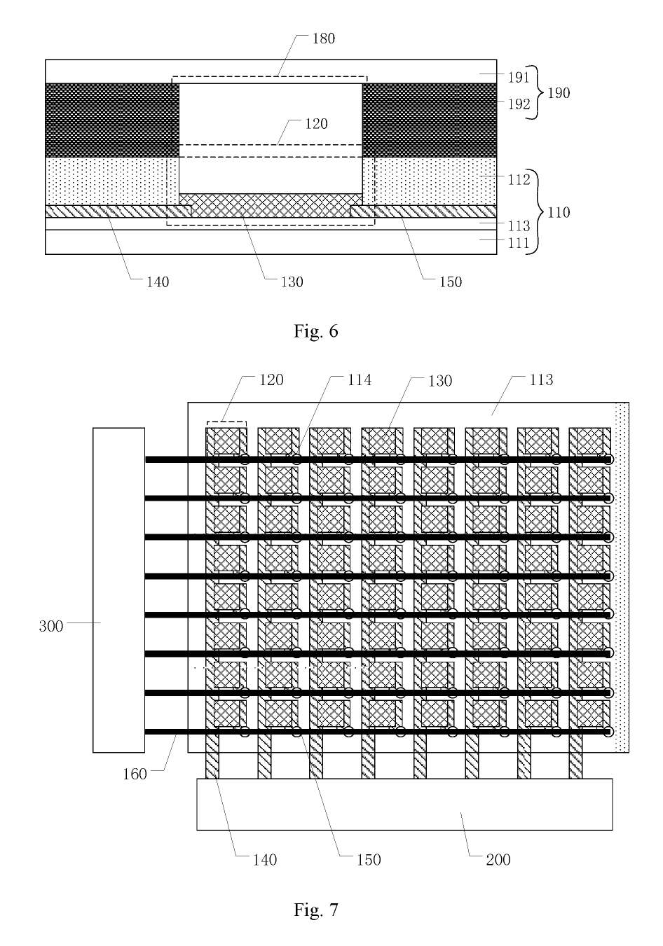

FIG. 6 is a schematic cross-sectional view of another gene sequencing chip according to an embodiment. As illustrated in FIG. 6, the gene sequencing chip further includes a second substrate 190 having a second surface 102. The second substrate 190 is arranged opposite to the first substrate 110 to form a cell, and the first surface 101 is arranged opposite to the second surface 102, so that the second substrate can be cell-assembled with the first substrate, thereby isolating the recessed portion from the outside, and improving sequencing precision and accuracy of the gene sequencing chip.

For example, the first substrate and the second substrate may be bonded together by a bonding method.

For example, in some examples, as illustrated in FIG. 6, the second substrate 190 further includes at least one flow channel 180, and the at least one flow channel 180 is arranged opposite to and in communication with the at least one recessed portion 120; therefore, various reagents can be applied to the recessed portion 120 through the flow channel 180. As illustrated in FIG. 6, the second substrate 190 further includes a flow channel sidewall 192 configured to define the flow channel 180, and a second substrate 191 located on a side of the flow channel 180 away from the second surface 102 and configured to support the flow channel sidewall 192.

For example, a material of the sidewall of the flow channel may be a resin, polydimethylsiloxane (PDMS), or the like.

For example, the second substrate may further include an inlet and an outlet connected with the flow channel to add and discharge the various reagents through the inlet and the outlet. For example, the inlet and outlet described above can be formed in the second substrate by a laser drilling method.

An embodiment of the present disclosure further provides a gene sequencing device. The gene sequencing device may include any of the gene sequencing chips as described above; therefore, the gene sequencing device including the gene sequencing chip also has technical effects corresponding to the beneficial technical effects of the gene sequencing chip. For example, the gene sequencing device can detect a change in resistance of the graphene oxide layer by the first electrode and the second electrode, so as to determine whether the currently added fluorescently labeled deoxy-ribonucleoside triphosphate is base paired with the sample to be tested or not, thereby directly converting the fluorescence information into an electrical signal for analysis, simplifying sequencing process, reducing sequencing time, and improving sequencing efficiency. Moreover, when the gene sequencing device is used for gene sequencing, an additional imaging device such as a CCD image sensor is not required, and the sequencing cost can be reduced.

FIG. 7 is a schematic plan view of a gene sequencing device according to an embodiment. The gene sequencing device includes a gene sequencing chip 100, a first circuit board 200, and a second circuit board 300. The first circuit board 200 is electrically connected with the first electrode 140, and the second circuit board 300 is electrically connected with the second electrode 150. For example, as illustrated in FIG. 7, when the gene sequencing chip includes the third electrode 160, the second circuit board 300 is electrically connected with the third electrode 160 so as to be electrically connected with the second electrode 150. The first circuit board 200 is configured to apply a driving signal to the first electrode 140, the second circuit board 300 is configured to detect a detection signal on the second electrode 150; or the second circuit board 300 is configured to apply a driving signal to the second electrode 150, and the first circuit board 200 is configured to detect a detection signal on the first electrode 140, so that detection of resistance of the graphene oxide layer 130 in each recessed portion can be achieved.

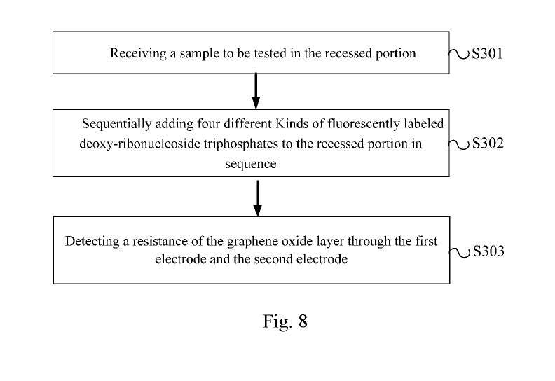

An embodiment of the present disclosure provides a sequencing method of a gene sequencing chip, and the gene sequencing chip may be any of the gene sequencing chips as described above. FIG. 8 is a flow chart of a sequencing method of a gene sequencing chip according to an embodiment. As illustrated in FIG. 8, the sequencing method includes steps S301-S303.

Step S301: receiving a sample to be tested in the recessed portion.

For example, a single strand of DNA to be tested can be placed in the recessed portion.

Step S302: adding a fluorescently labeled deoxy-ribonucleoside triphosphate to the recessed portion. For example, four different kinds of fluorescently labeled deoxy-ribonucleoside triphosphates are added to the recessed portion in sequence.

Step S303: detecting a resistance of the graphene oxide layer through the first electrode and the second electrode.

In the sequencing method of the gene detection chip provided by the embodiment, a sample to be tested (for example, a single strand of DNA) is placed in the microstructure, and four fluorescently labeled deoxy-ribonucleoside triphosphates (dNTPs) are added to the microstructure, if a certain fluorescently labeled deoxy-ribonucleoside triphosphate is base paired with the sample to be tested, both the fluorescent dye and the graphene oxide have a C═C—C═C conjugated double bond and a distance therebetween is very close, resulting in generating a stack of C═C—C═C, resonance and transfer of fluorescence energy, i.e., fluorescence quenching, occur. When resonance and transfer of fluorescence occur, the photon energy generated by the fluorescent dye is absorbed by the graphene oxide, thereby reducing a resistance of the graphene oxide. The sequencing method can detect the change in resistance of the graphene oxide layer by the first electrode and the second electrode, so that whether the currently added fluorescently labeled deoxy-ribonucleoside triphosphate has a base paired reaction with the sample to be tested can be determined, thereby directly converting fluorescence information into electrical signals for analysis, simplifying sequencing process, reducing sequencing time, and improving sequencing efficiency. Moreover, the sequencing method does not require an additional camera device such as a CCD image sensor, thereby reducing sequencing cost. It should be noted that the above-mentioned four different kinds of deoxy-ribonucleoside triphosphates include adenine deoxy-ribonucleoside triphosphate, uracil deoxy-ribonucleoside triphosphate, cytosine deoxy-ribonucleoside triphosphate, and guanine deoxy-ribonucleoside triphosphate.

For example, in some examples, the four different kinds of deoxy-ribonucleoside triphosphates described above are reversibly terminated deoxy-ribonucleoside triphosphates, and the sequencing method further includes: washing the reversible terminated deoxy-ribonucleoside triphosphate added in the reaction cell, and adding a sulfhydryl reagent. After a detection of a type of the base at a previous position on the sample to be tested (for example, a single strand of DNA) is completed, it is necessary to wash away the reversible terminated deoxy-ribonucleoside triphosphate added in the reaction cell and add a sulfhydryl reagent.

With respect to the present disclosure, the following statements should be noted.

(1) The accompanying drawings involve only the structure(s) in connection with the embodiment(s) of the present disclosure, and other structure(s) can be referred to common design(s).

(2) In case of no conflict, features in one embodiment or in different embodiments can be combined.

What have been described above are only specific implementations of the present disclosure, the protection scope of the present disclosure is not limited thereto. Any modifications or substitutions easily occur to those skilled in the art within the technical scope of the present disclosure should be fallen within the protection scope of the present disclosure. Therefore, the protection scope of the present disclosure should be based on the protection scope of the claims.

1. A gene sequencing chip, comprising:
a first substrate having a first surface;
at least one recessed portion recessed from the first surface into the first substrate;
a graphene oxide layer located at a bottom of the recessed portion; and
a first electrode and a second electrode electrically connected with the graphene oxide layer, respectively,
wherein the recessed portion is configured to receive a sample to be tested, and the first electrode and the second electrode are configured to detect a resistance of the graphene oxide layer.

2. The gene sequencing chip according to claim 1, wherein the first electrode is not in direct contact with the second electrode.

3. The gene sequencing chip according to claim 2, wherein the first substrate comprises:
a sidewall of the recessed portion configured to define the recessed portion; and
a first base substrate located on a side of the recessed portion away from the first surface and configured to support the sidewall of the recessed portion,
wherein the first electrode and the second electrode are located between the sidewall of the recessed portion and the first base substrate, the first electrode and the second electrode extend to the bottom of the recessed portion to be in contact with the graphene oxide layer, respectively.

4. The gene sequencing chip according to claim 3, wherein the first electrode comprises a first contact portion extending to the recessed portion, the second electrode comprises a second contact portion extending to the recessed portion, and the graphene oxide layer covers the first contact portion and the second contact portion.

5. The gene sequencing chip according to claim 3, wherein a shape of an orthographic projection of the recessed portion on the first substrate comprises two opposite sidewalls, and the first electrode and the second electrode are located between the two opposite sidewalls of the recessed portion and the first base substrate, respectively.

6. The gene sequencing chip according to claim 3, wherein the first substrate further comprises
an insulation layer located on a side of the first base substrate close to the first surface, and the insulation layer is located on a side of the first electrode, the second electrode, and the graphene oxide layer away from the first surface.

7. The gene sequencing chip according to claim 1, wherein the recessed portion comprises a microchannel or a micro-hole.

8. The gene sequencing chip according to claim 1, wherein the at least one recessed portion comprises a plurality of recessed portions, graphene oxide layers are provided in a one-to-one correspondence with the plurality of recessed portions.

9. The gene sequencing chip according to claim 8, wherein the plurality of recessed portions are arranged in an array, and the graphene oxide layers in the recessed portions of a same column share one first electrode.

10. The gene sequencing chip according to 8, further comprising
a third electrode electrically connected with second electrodes in the recessed portions of a same row.

11. The gene sequencing chip according to claim 10, wherein the first substrate comprises a plurality of connection via holes, and the third electrode is electrically connected with the second electrodes in the recessed portions of the same row through the plurality of connection via holes.

12. The gene sequencing chip according to claim 11, wherein the third electrode is located on the first surface.

13. The gene sequencing chip according to claim 1, further comprising:
a second substrate having a second surface,
wherein the second substrate and the first substrate are opposite to each other to form a cell, and the first surface is opposite to the second surface.

14. The gene sequencing chip according to claim 13, wherein the second substrate further comprises:
at least one flow channel, the at least one flow channel being located opposite to and in communication with the at least one recessed portion;
a second base substrate located on a side of the flow channel away from the second base surface; and
a flow channel sidewall located on a side of the second substrate close to the second surface, the flow channel sidewall being configured to define the flow channel.

15. A gene sequencing device comprising the gene sequencing chip according to claim 1.

16. The gene sequencing device according to claim 15, further comprising:
a first circuit board electrically connected with the first electrode; and
a second circuit board electrically connected with the second electrode,
wherein the first circuit board is configured to apply a driving signal to the first electrode, the second circuit board is configured to detect a detection signal on the second electrode, or the second circuit board is configured to apply a driving a signal to the second electrode, the first circuit board is configured to detect a detection signal on the first electrode.

17. A sequencing method of a gene sequencing chip, wherein the gene sequencing chip comprises the gene sequencing chip according to claim 1, the sequencing method comprising:
receiving a sample to be tested in the recessed portion;
adding a fluorescently labeled deoxy-ribonucleoside triphosphate to the recessed portion; and
detecting a resistance of the graphene oxide layer through the first electrode and the second electrode.
